(19) United States
(12) Patent Application Publication
(10) Pub. No.: US 2020/0369652 A1
(43) Pub. Date: Nov. 26, 2020

(54) PROCESSES FOR PREPARATION OF LIFITEGRAST AND INTERMEDIATES THEREOF

(71) Applicant: DR. REDDY'S LABORATORIES LIMITED, Hyderabad (IN)

(72) Inventors: Srinivas ACHANTA, Noida (IN); Ramesh CHAKKA, Hyderabad (IN); Vilas Hareshwar DAHANUKAR, Hyderabad (IN); Elati Ravi Rama CHANDRASEKHAR, Hyderabad (IN); Srinivasa Reddy BHIMAVARAPU, Hyderabad (IN); Mahendar MADARABOINA, Hyderabad (IN)

(21) Appl. No.: 16/636,212

(22) PCT Filed: Aug. 2, 2018
(86) PCT No.: PCT/IB2018/055801

§ 371 Date: Feb. 3, 2020

(30) Foreign Application Priority Data
Aug. 3, 2017 (IN) 201741027624

(51) Int. Cl. C07D 405/10

(52) U.S. Cl. CPC C07D 405/10

(57) ABSTRACT

The present invention provides process for preparation of lifitegrast, amine salts of lifitegrast, process for preparation of amine salts of lifitegrast and their use in purification of lifitegrast, and also provided are process for preparation of amorphous lifitegrast and pharmaceutical compositions comprising lifitegrast prepared by the processes of the present invention.

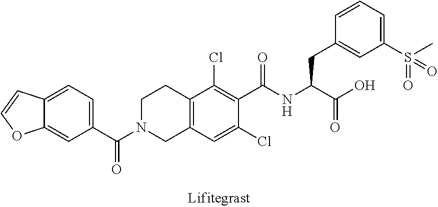 [SMILES: CS(=O)(=O)c1cccc(C[C@H](NC(=O)c2c(Cl)cc3c(c2Cl)CCN(C(=O)c2ccc4ccoc4c2)C3)C(=O)O)c1]

### CROSS-REFERENCE TO RELATED APPLICATIONS

This application claims the benefit of Indian provisional patent application No. 201741027624 filed on 3 Aug. 2017.

### FIELD OF THE INVENTION

The present application relates to processes for preparation of Lifitegrast and intermediates thereof.

### BACKGROUND OF THE INVENTION

The drug compound having the adopted name Lifitegrast, has a chemical name (S)-2-(2-(benzofuran-6-carbonyl)-5,7-dichloro-1,2,3,4-tetrahydroisoquinoline-6-carboxamido)-3-(3-(methylsulfonyl)phenyl)propanoic acid, and is represented by the structure of formula I.

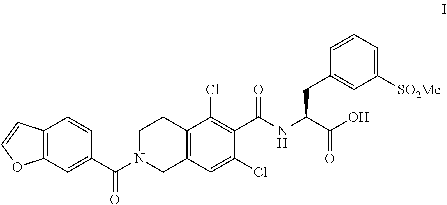 [SMILES: CS(=O)(=O)c1cccc(C[C@H](NC(=O)c2c(Cl)cc3c(c2Cl)CCN(C(=O)c2ccc4ccoc4c2)C3)C(=O)O)c1]

Lifitegrast is an inhibitor of Lymphocyte Function-Associated Antigen (LFA-1) and is used for the treatment of dry eye disease (DED).

Lifitegrast is described in U.S. Pat. No. 7,314,938 B2 (US '938) and in U.S. Pat. No. 8,084,047 B2 (US '047). However, neither the US '938 patent nor the US '047 patent describe process for preparation of lifitegrast specifically.

U.S. Pat. No. 8,080,562 B2 (US '562) describes a process for preparation of lifitegrast. The process described in US '562 is schematically represented below.

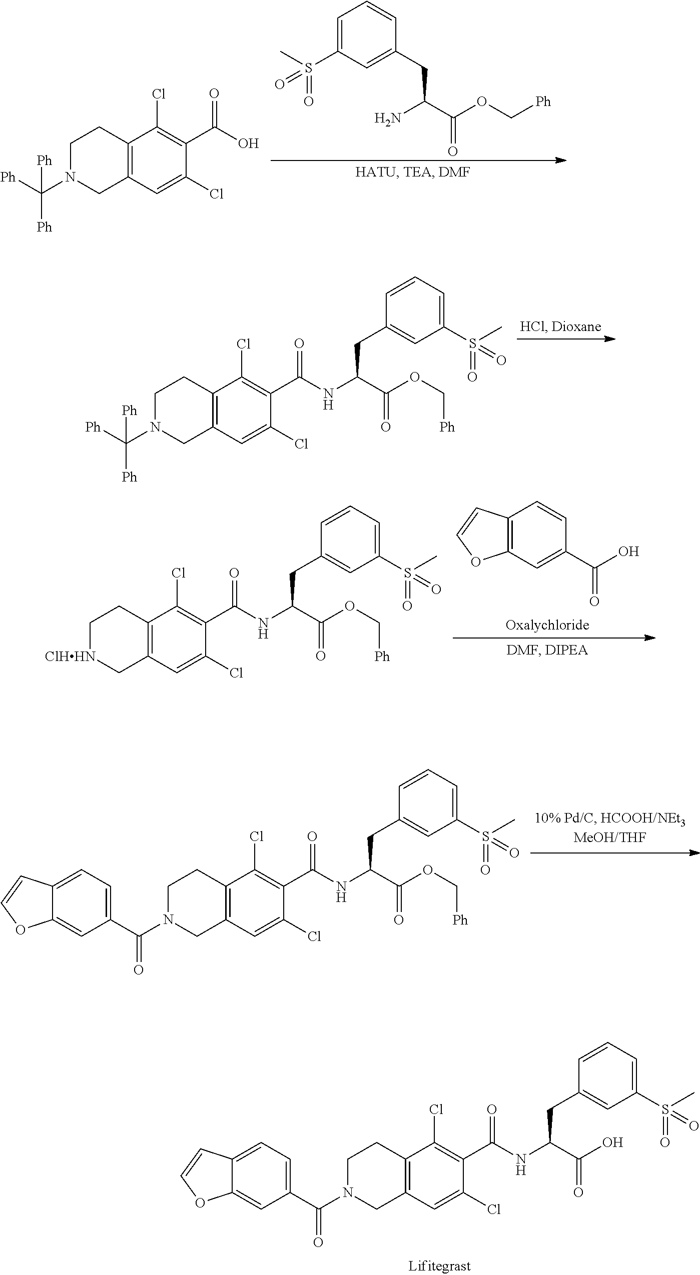

U.S. Pat. No. 8,378,105 B2 (US '105) describes a process for preparation of lifitegrast. The process described in US '105 is schematically represented below.

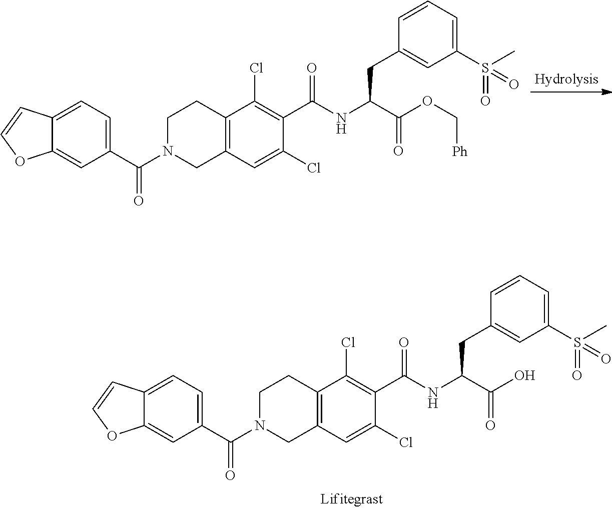 [SMILES: CS(=O)(=O)c1cccc(C[C@H](NC(=O)c2c(Cl)cc3c(c2Cl)CCN(C(=O)c2ccc4ccoc4c2)C3)C(=O)OCc2ccccc2)c1 ; CS(=O)(=O)c1cccc(C[C@H](NC(=O)c2c(Cl)cc3c(c2Cl)CCN(C(=O)c2ccc4ccoc4c2)C3)C(=O)O)c1]

U.S. Pat. No. 9,085,553 B2 (US '553) describes a process for preparation of lifitegrast. The process described in US '553 is schematically represented below.

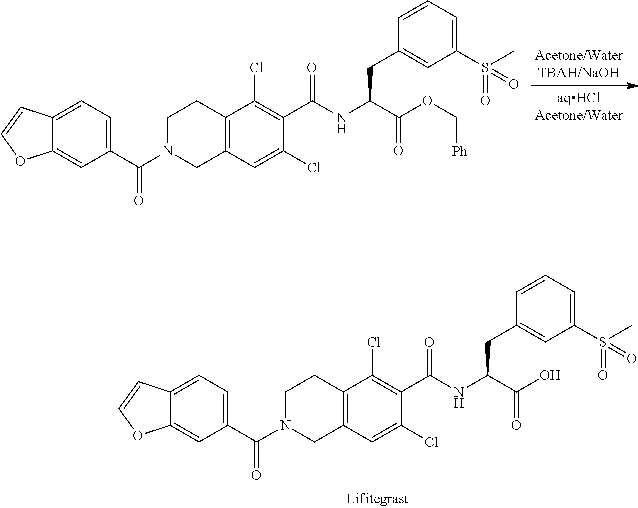 [SMILES: CS(=O)(=O)c1cccc(C[C@H](NC(=O)c2c(Cl)cc3c(c2Cl)CCN(C(=O)c2ccc4ccoc4c2)C3)C(=O)OCc2ccccc2)c1 ; CS(=O)(=O)c1cccc(C[C@H](NC(=O)c2c(Cl)cc3c(c2Cl)CCN(C(=O)c2ccc4ccoc4c2)C3)C(=O)O)c1]

The prior-art processes herein described above suffer from drawbacks such as more process steps and low purity. Hence there is a continuing need to develop simplified and improved process for preparing Lifitegrast which is suitable for commercial manufacturing in high purity and yield.

### SUMMARY OF THE INVENTION

The present application provides processes for preparation of Lifitegrast and its intermediates thereof.

In the first aspect, the present application provides a process for preparation of Lifitegrast comprising reacting compound of formula VI with compound of formula II in presence of an activating agent and a suitable base.

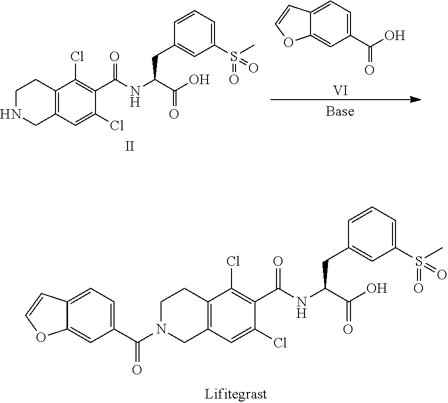

In the second aspect, the present application provides a process for preparation of Lifitegrast comprising:
(a) reacting compound of formula V with a compound of formula VII in presence of suitable activating agent and a base

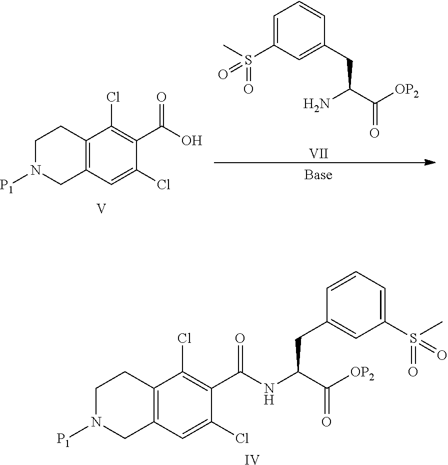

(b) hydrolyzing the compound of formula IV using a suitable base or a suitable acid to form a compound of formula II

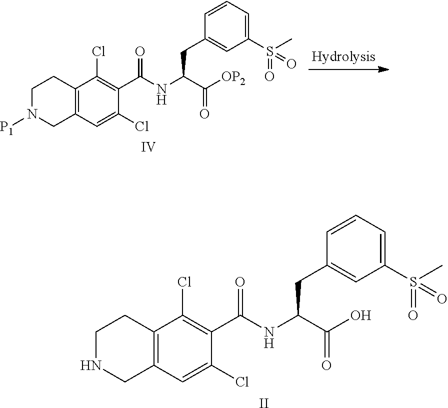

(c) reacting compound of formula II with compound of formula VI in presence of a suitable activating agent and a suitable base to form Lifitegrast.

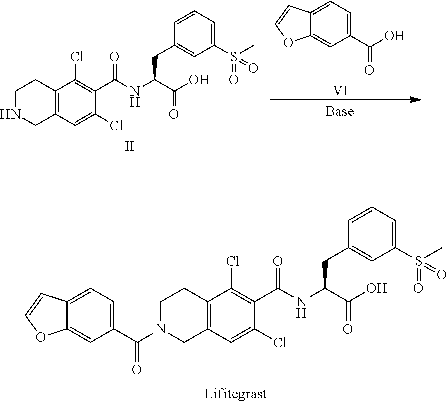

wherein P1 and P2 are protecting groups.

In the third aspect, the present application provides a process for preparation of Lifitegrast comprising reacting compound of formula VIII with a compound of formula VII-A in presence of a suitable base.

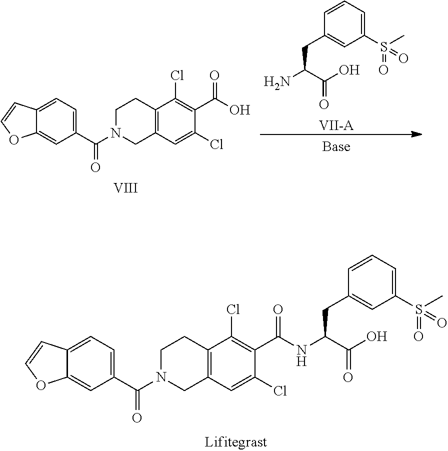

In the fourth aspect, the present application provides a process for preparation of Lifitegrast comprising:
(a) reacting compound of formula V-A with a compound of formula VI in the presence of a suitable activating agent and a suitable base to form a compound of formula

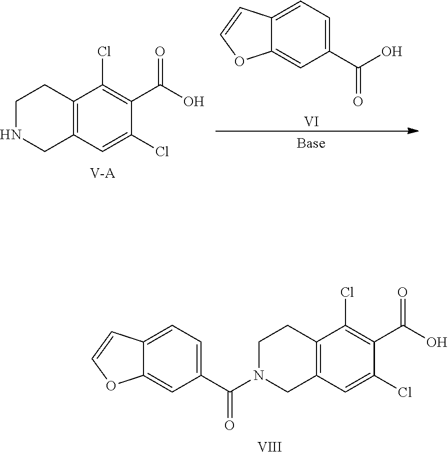

(b) reacting the compound of formula VIII with a compound of formula VII-A in presence of a suitable activating agent and a suitable base to form Lifitegrast.

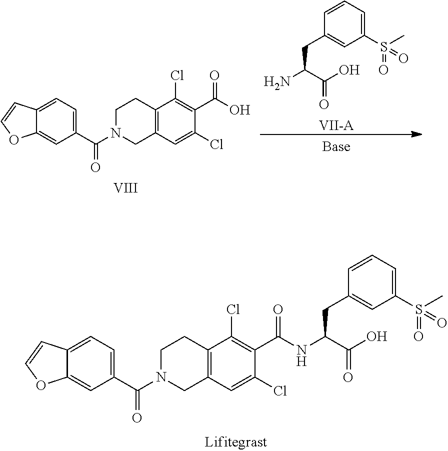

In the fifth aspect, the present application provides a process for purification of Lifitegrast comprising:
(a) mixing Lifitegrast, an amine and a solvent to provide an amine salt of lifitegrast
(b) optionally isolating the amine salt of lifitegrast, and
(c) isolating pure lifitegrast from the amine salt of lifitegrast.

In the sixth aspect, the present application provides a process for preparation of amorphous lifitegrast, comprising,
(a) providing a solution of lifitegrast in a suitable solvent;
(b) optionally, treating the solution of obtained in step (a) with a base;
(c) treating the solution of step (a) or step (b) with an acid; and
(d) isolating amorphous lifitegrast from the acidic solution of step (c).

In the seventh aspect, the present application provides Lifitegrast containing less than 1% w/w of its (R)-isomer.

In the eighth aspect, the present application provides Lifitegrast containing less than 0.5% w/w of its (R)-isomer.

In the ninth aspect, the present application provides Lifitegrast containing less than 0.1% w/w of its (R)-isomer.

In the tenth aspect, the present application provides a pharmaceutical composition comprising lifitegrast prepared by the process of the present invention and at least one pharmaceutically acceptable carrier.

### BRIEF DESCRIPTION OF THE DRAWINGS

FIG. 1 is a PXRD pattern of amorphous Lifitegrast prepared by the process of example-8

FIG. 2 is a PXRD pattern of crystalline Lifitegrast dicyclohexyl amine salt prepared by the process of example-10

FIG. 3 is a PXRD pattern of crystalline Lifitegrast prepared by the process of example-12(A)

FIG. 4 is a PXRD pattern of crystalline Lifitegrast prepared by the process of example-12(B).

### DETAILED DESCRIPTION OF THE INVENTION

The present application provides processes for preparation of Lifitegrast and intermediates thereof.

In the first aspect, the present application provides a process for preparation of Lifitegrast comprising reacting compound of formula VI with compound of formula II in presence of suitable base

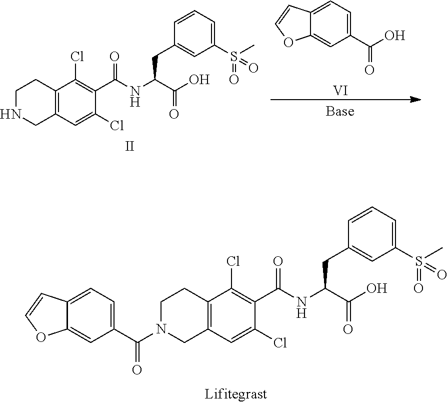

The compound of formula II and formula VI are known compounds and may be obtained by any process including the process described in the art or by the process described in this application. These starting materials may be purified by the processes known in the art to get the desired purity before proceeding for the reaction.

The process involves coupling of compound of formula II with a compound of formula VI using a suitable activating agent, a suitable base and a suitable solvent.

The acid activating agent that may be used is thionyl chloride, oxalyl chloride, dicyclohexyl carbodiimide and the like.

The base to be used is for example an inorganic base such as sodium hydroxide, lithium hydroxide, potassium hydroxide and the like, or an organic base such as triethylamine, N,N-diisopropylethylamine (DIPEA), pyridine, piperidine, 1,8-Diazabicyclo[5.4.0[undec-7-ene (DBU), 4-Dimethylaminopyridine (DMAP). The solvent to be used for example acetone, acetonitrile, ethylacetate, dimethylformamide, N-methylpyrrolidine, dimethylsulfoxide, tetrahydrofuran, chlorobenzene and toluene.

The reaction optionally be carried out in presence of a suitable coupling agent such as (1-[Bis(dimethylamino)methylene]-1H-1,2,3-triazolo[4,5-b] pyridinium oxide hexafluorophosphate (HATU), 1-hydroxybenzotriazole (HOBT), hydroxyazabenzotriazole (HOAT), hydroxy succinimide (HOSu), and N-(3-dimethylaminopropyl)-N′-ethylcarbodiimide hydrochloride (EDC.HCl).

The reaction temperature may vary depending on the kinds of the solvent and the base used, but may be generally within a range of from about 0° C. to about 100° C. The reaction time varies depending on the solvent and base used and on the reaction temperature, but may be generally within a range of from about 5 minutes to about 20 hours. After completion of the reaction the reaction mass may be poured into water optionally containing acid and the aqueous mass is extracted with a suitable water immiscible solvent such as ethylacetate followed by washings and the organic layer may be concentrated to isolate Lifitegrast or lifitegrast may be filtered from the organic layer.

In the second aspect, the present application provides a process for preparation of Lifitegrast comprising:
(a) reacting compound of formula V with a compound of formula VII in presence of suitable activating agent and a base

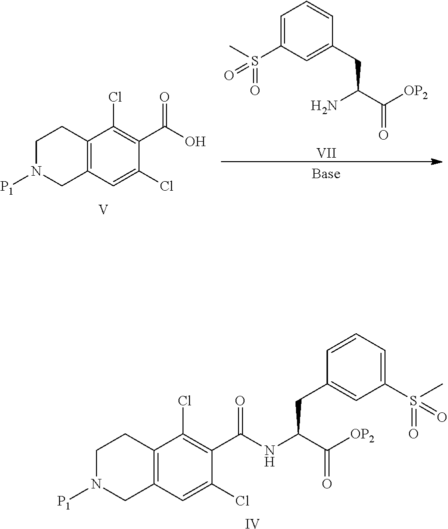

(b) hydrolyzing the compound of formula IV using a suitable base or a suitable acid to form a compound of formula II

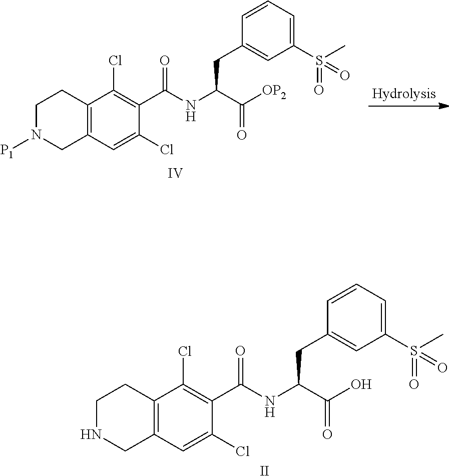

(c) reacting compound of formula II with compound of formula VI in presence of a suitable activating agent and a suitable base to form Lifitegrast.

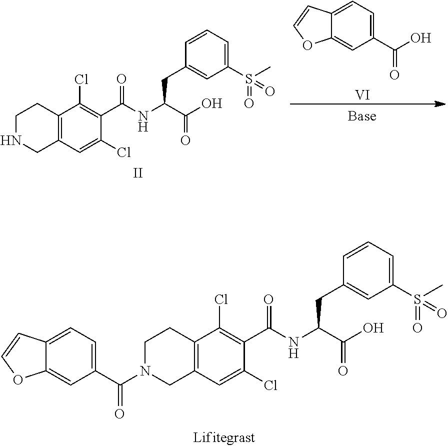

wherein P1 and P2 are protecting groups.

The step (a) of the process involves reaction of a compound of formula V with a compound of formula VII in presence of a suitable base to form a compound of formula IV.

The P₁ and P₂ are independently selected from the group comprising of 9-Fluorenylmethyloxy carbonyl (FMOC), tert-butyloxycarbonyl (BOC), benzyloxy carbonyl (CBZ), acetyl, trifluoroacetyl, benzyl, triphenylmethyl (trityl), and p-toluenesulfonyl. In one embodiment the P₁ is triphenylmethyl, and P₂ is benzyl.

The compound of formula V and formula VII are known compounds and may be obtained by any process including the process described in the art or by the process described in this application. These starting materials may be purified by the processes known in the art to get the desired purity before proceeding for the reaction.

The reaction is carried out in the presence of a suitable base and a suitable solvent. The base to be used is for example an organic base such as triethylamine, N,N-diisopropylethylamine (DIPEA), pyridine, piperidine, (1,8-Diazabicyclo[5.4.0[undec-7-ene (DBU), 4-Dimethylaminopyridine (DMAP).

The reaction optionally be carried out in presence of a suitable coupling agent such as (1-[Bis(dimethylamino)methylene]-1H-1,2,3-triazolo[4,5-b] pyridinium oxide hexafluorophosphate (HATU), 1-hydroxybenzotriazole (HOBT), hydroxyazabenzotriazole (HOAT), hydroxy succinimide (HOSu), and N-(3-dimethylaminopropyl)-N′-ethylcarbodiimide hydrochloride (EDC.HCl)

The solvent to be used for example acetone, acetonitrile, ethylacetate, isopropyl acetate, dimethylformamide, N-methylpyrrolidine, dimethylsulfoxide, tetrahydrofuran and toluene.

The amount of compound of formula VII to be used may be generally within a range of 0.5 to 2.0 molar ratio relative to the compound of formula V. The reaction temperature may vary depending on the kinds of the solvent and the base used, but may be generally within a range of from about 0° C. to about 100° C. The reaction time varies depending on the solvent and base used and on the reaction temperature, but may be generally within a range of from about 1 hour to about 20 hours. After completion of the reaction the reaction mass may be poured into water and the aqueous mass is extracted with a suitable water immiscible solvent to isolate the compound of formula IV.

The step (b) of the process involves hydrolysis of compound of formula IV using a suitable base or a suitable acid to form a compound of formula II. The base hydrolysis may be carried out using a suitable base and a suitable solvent. The base used for hydrolysis is for example an inorganic base such as sodium hydroxide, lithium hydroxide, potassium hydroxide, sodium bicarbonate and potassium bicarbonate. The solvent to be used for example methanol, ethanol, acetone tetrahydrofuran, dioxane and water or a mixture thereof. If the P₁ and P₂ are different, base hydrolysis is carried out first and then acid hydrolysis is carried out.

In one embodiment, with the base hydrolysis the compound of formula IV is converted into compound of formula III as shown in the below scheme.

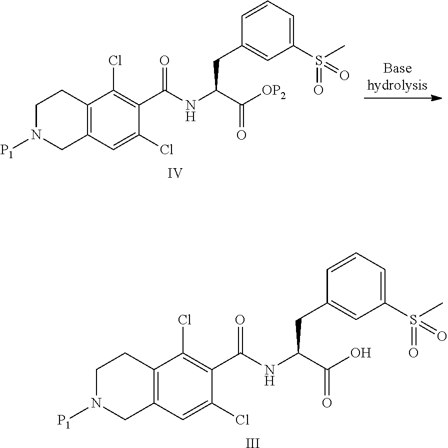

After the base hydrolysis, the acid hydrolysis may be carried out in-situ without isolating the compound of formula III. The acid hydrolysis is carried out using a suitable acid and a suitable solvent. The acid used for hydrolysis is for example hydrochloric acid, acetic acid and trifluoroacetic acid and the like. The acid hydrolysis may be carried out using an aqueous media.

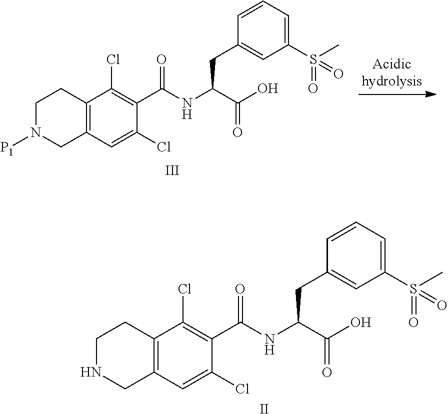

After acidic hydrolysis the compound may be isolated from the aqueous layer or may be extracted into an organic solvent such as ethyl acetate. The aqueous layer or the organic layer containing the compound of formula II may be used directly in the next step.

In an embodiment hydrolysis of compound of formula IV using a suitable base or suitable acid may directly give the compound of formula II.

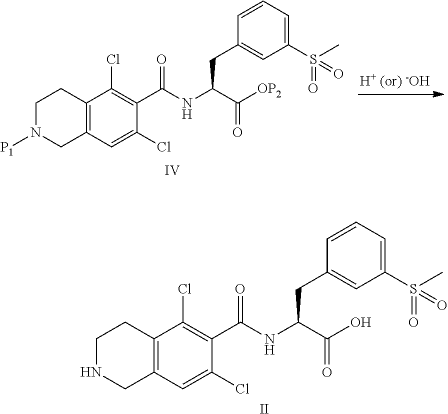

Step (c) involves coupling of compound of formula II with a compound of formula VI using a suitable coupling agent, a suitable base and a suitable solvent. The acid activating agent that may be used is thionyl chloride, oxalyl chloride, dicyclohexyl carbodiimide, acetic anhydride and the like.

The base to be used is for example an inorganic base such as sodium hydroxide, lithium hydroxide, potassium hydroxide and the like, or an organic base such as triethylamine, Diisopropylethylamine (DIPEA), pyridine, piperidine, 1,8-Diazabicyclo[5.4.0[undec-7-ene (DBU), 4-Dimethylaminopyridine (DMAP). The solvent to be used for example acetone, acetonitrile, ethylacetate, dimethylformamide, N-methylpyrrolidine, dimethylsulfoxide, tetrahydrofuran, chlorobenzene and toluene.

The reaction optionally be carried out in presence of a suitable coupling agent such as (1-[Bis(dimethylamino)methylene]-1H-1,2,3-triazolo[4,5-b] pyridinium oxide hexafluorophosphate (HATU), 1-hydroxybenzotriazole (HOBT), hydroxyazabenzotriazole (HOAT), and hydroxy succinimide (HOSu).

The amount of compound of formula VI to be used may be generally within a range of 0.8 to 3.0 molar ratio relative to the compound of formula II. The reaction temperature may vary depending on the kinds of the solvent and the base used, but may be generally within a range of from about 30° C. to about 60° C. The reaction time varies depending on the solvent and base used and on the reaction temperature, but may be generally within a range of from about 1 hour to about 20 hours. After completion of the reaction the reaction mass may be poured into water optionally containing an acid and the aqueous mass is extracted with a suitable water immiscible solvent followed by washings and the organic layer may be concentrated to isolate Lifitegrast.

In the third aspect, the present application provides a process for preparation of Lifitegrast comprising reacting compound of formula VIII with a compound of formula VIl-A in presence of a suitable activating agent and a suitable base.

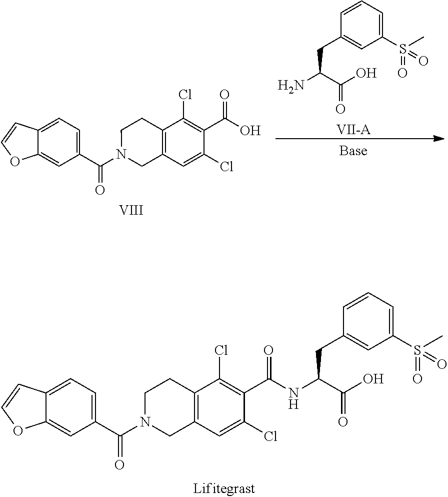

The compound of formula VII-A and formula VIII are known compounds and may be obtained by any process including the process described in the art or by the process described in this application. These starting materials may be purified by the processes known in the art to get the desired purity before proceeding for the reaction.

The process involves coupling of compound of formula VIII with a compound of formula VII-A using a suitable acid activating agent, a suitable base and a suitable solvent. The acid activating agent that may be used is thionyl chloride, oxalyl chloride, acetic anhydride, dicyclohexyl carbodiimide, N-(3-dimethylaminopropyl)-N′-ethylcarbodiimide hydrochloride (EDC.HCl) and the like.

The base to be used is for example an inorganic base such as sodium hydroxide, lithium hydroxide, potassium hydroxide and the like, or an organic base such as triethylamine, N,N-diisopropylethylamine (DIPEA), pyridine, piperidine, 1,8-Diazabicyclo[5.4.0[undec-7-ene (DBU), 4-Dimethylaminopyridine (DMAP). The solvent to be used for example acetone, acetonitrile, ethylacetate, dimethylformamide, N-methylpyrrolidine, dimethylsulfoxide, tetrahydrofuran, chlorobenzene and toluene.

The reaction optionally be carried out in presence of a suitable coupling agent such as (1-[Bis(dimethylamino)methylene]-1H-1,2,3-triazolo[4,5-b] pyridinium oxide hexafluorophosphate (HATU), 1-hydroxybenzotriazole (HOBT), hydroxyazabenzotriazole (HOAT), and hydroxy succinimide (HOSu).

The amount of compound of formula VII-A to be used may be generally within a range of 0.8 to 3.0 molar ratio relative to the compound of formula VIII. The reaction temperature may be generally within a range of from about 0° C. to about 100° C. The reaction time varies depending on the solvent and base used and on the reaction temperature, but may be generally within a range of from about 15 minutes to about 20 hours. After completion of the reaction the reaction mass may be poured into water optionally containing an acid and the aqueous mass is extracted with a suitable water immiscible solvent followed by washings and the organic layer may be concentrated to isolate Lifitegrast.

In the fourth aspect, the present application provides a process for preparation of Lifitegrast comprising:
(a) reacting compound of formula V-A with a compound of formula VI in the presence of a suitable base to form a compound of formula

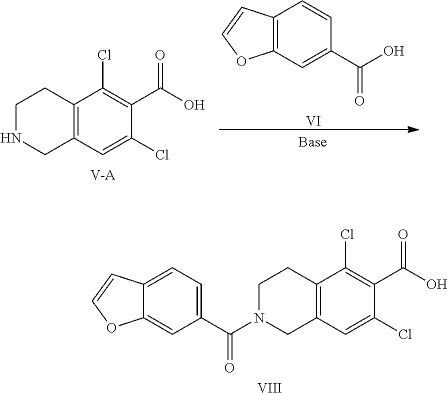

(b) reacting the compound of formula VIII with a compound of formula VII-A in presence of suitable base to form Lifitegrast.

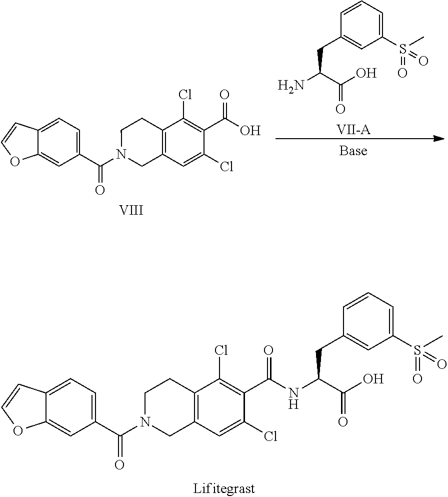

The compound of formula V-A, VI and formula VII-A are known compounds and may be obtained by any process including the process described in the art or by the process described in this application. These starting materials may be purified by the processes known in the art to get the desired purity before proceeding for the reaction.

The step (a) involves reaction of compound of formula V-A with compound of formula VI. Before reacting the compound of formula VI is converted into an acid chloride by reacting it with a chlorinating agent such as thionyl chloride, oxalyl chloride, dicyclohexyl carbodiimide, acetic anhydride and the like. Then the coupling reaction is carried out in the presence of a suitable base and a suitable solvent. The base to be used is for example an organic base such as triethylamine, N,N-diisopropylethylamine (DIPEA), pyridine, piperidine, 1,8-Diazabicyclo[5.4.0[undec-7-ene (DBU), 4-Dimethylaminopyridine (DMAP). The solvent to be used for example acetone, acetonitrile, ethylacetate, dimethylformamide, dimethylacetamide, N-methylpyrrolidine, dimethylsulfoxide, tetrahydrofuran and toluene or any mixture thereof.

The reaction temperature may vary depending on the kinds of the solvent and the base used, but may be generally within a range of from about −10° C. to about 50° C. The reaction time varies depending on the solvent and base used and on the reaction temperature, but may be generally within a range of from about 10 minutes to about 20 hours. After completion of the reaction the reaction mass may be poured into water optionally containing an acid and the aqueous mass is extracted with a suitable water immiscible solvent followed by optional washings and the organic layer may be concentrated to isolate the compound of formula VIII.

The step (b) involves coupling of compound of formula VIII with a compound of formula VII-A using a suitable acid activating agent, a suitable base and a suitable solvent. The acid activating agent that may be used is thionyl chloride, oxalyl chloride, dicyclohexyl carbodiimide, acetic anhydride and the like.

The base to be used is for example an inorganic base such as sodium hydroxide, lithium hydroxide, potassium hydroxide and the like, or an organic base such as triethylamine, N,N-diisopropylethylamine (DIPEA), pyridine, piperidine, 1,8-Diazabicyclo[5.4.0[undec-7-ene (DBU), 4-Dimethylaminopyridine (DMAP). The solvent to be used for example acetone, acetonitrile, ethylacetate, dimethylformamide, N-methylpyrrolidine, dimethylsulfoxide, tetrahydrofuran, chlorobenzene and toluene.

The reaction optionally be carried out in presence of a suitable coupling agent such as (1-[Bis(dimethylamino)methylene]-1H-1,2,3-triazolo[4,5-b] pyridinium oxide hexafluorophosphate (HATU), 1-hydroxybenzotriazole (HOBT), hydroxyazabenzotriazole (HOAT), and hydroxy succinimide (HOSu).

The amount of compound of formula VII-A to be used may be generally within a range of 0.8 to 3.0 molar ratio relative to the compound of formula VIII. Optionally, small amount of water may be added to the reaction to enhance the solubility of the reagents. The reaction temperature may be generally within a range of from about 30° C. to about 60° C. The reaction time varies depending on the solvent and base used and on the reaction temperature, but may be generally within a range of from about 1 hour to about 20 hours. After completion of the reaction the reaction mass may be poured into water optionally containing an acid and the aqueous mass is extracted with a suitable water immiscible solvent followed by optional washing the organic layer the organic layer may be concentrated to isolate Lifitegrast.

In the fifth aspect, the present application provides a process for purification of Lifitegrast comprising:
(a) mixing Lifitegrast, an amine and a solvent to provide an amine salt of lifitegrast
(b) optionally isolating the amine salt of lifitegrast, and
(c) isolating pure lifitegrast from the amine salt of lifitegrast.

The process involves mixing lifitegrast with an amine. The amine may be an organic amine or an inorganic amine. The organic amine may be a primary amine such as methylamine, ethylamine cyclohexylamine, aniline and the like or a secondary amine such as dimethylamine, diethylamine, diisopropyl amine, dicyclohexylamine and the like or a tertiary amine such triethylamine, diisopropylethylamine and the like. The organic amine may be a chiral amine or achiral amine. In one embodiment the amine is dicyclohexylamine. Lifitegrast is mixed with the amine in a suitable solvent such as tetrahydrofuran, acetonitrile, dioxane, ethylacetate, methanol, ethanol, isopropanol, dichloromethane, chlorobenzene, toluene, water or a mixture thereof. The precipitated amine salt of lifitegrast may be isolated. The resulting salt may optionally be further dried.

In one embodiment, lifitegrast is mixed with dicyclohexylamine in a suitable solvent such as tetrahydrofuran, acetonitrile, dioxane, ethylacetate, water or a mixture thereof. The precipitated lifitegrast dicyclohexylamine salt may be isolated. The resulting salt may optionally be further dried.

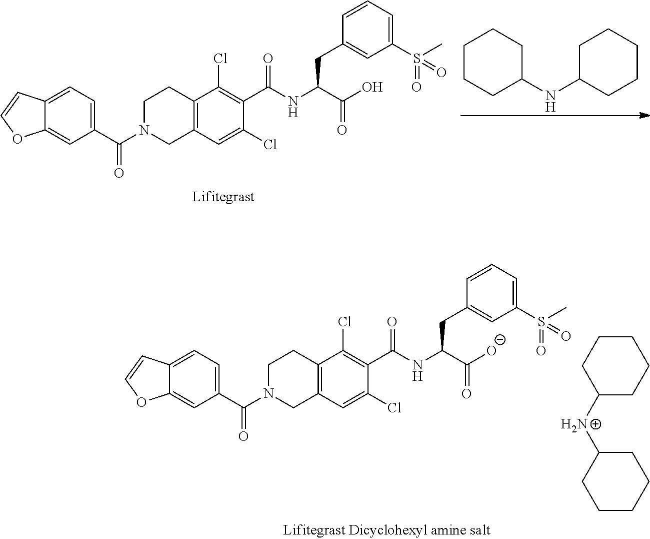

The lifitegrast dicyclohexyl amine salt may exist in amorphous form or in crystalline form. In one embodiment, the lifitegrast dicyclohexyl amine salt exists in crystalline form and a PXRD pattern of the lifitegrast dicyclohexyl amine salt is shown in FIG. 2.

The salt is then treated with a suitable acid such as aqueous hydrochloric acid, aqueous orthophosphoric acid, or aqueous sulphuric acid to isolate the pure lifitegrast. Pure lifitegrast may also be isolated from the aqueous layer or the aqueous layer containing the pure lifitegrast may be extracted with a suitable water immiscible solvent such as ethylacetate. The solvent layer is concentrated to get pure Lifitegrast.

The resulting compound may optionally be further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 60° C., less than about 50° C., less than about 40° C., less than about 30° C., less than about 20° C., or any other suitable temperatures; at atmospheric pressure or under a reduced pressure; as long as the Lifitegrast is not degraded in its quality. The drying can be carried out for any desired times until the required product quality is achieved. Suitable time for drying can vary from few minutes to several hours for example from about 30 minutes to about 24 or more hours.

Lifitegrast purified by the process of the present invention may exist in amorphous form or in crystalline form.

In an embodiment Lifitegrast can be purified using the above similar approach of forming Lifitegrast salt with any base, followed by optional purification of the salt, and converting the salt into Lifitegrast by the methods known in the art.

Lifitegrast purified by the process of amine salt formation leads to enrichment of (S)-isomer and reduces the content of (R)-isomer.

Lifitegrast purified by the process of the present application contains less than 1% w/w of its (R)-isomer. In another embodiment Lifitegrast purified by the process of the present application contains less than 0.5% w/w of its (R)-isomer. In another embodiment, Lifitegrast purified by this process contains less than 0.2% w/w of its (R)-isomer. In another embodiment, Lifitegrast purified by this process contains less than 0.05% w/w of its (R)-isomer.

In another aspect, the present application provides a process for preparation of amorphous lifitegrast, comprising,
(a) providing a solution of lifitegrast in a suitable solvent;
(b) optionally, treating the solution of obtained in step (a) with a base;
(c) treating the solution of step (a) or step (b) with an acid; and
(d) isolating amorphous lifitegrast from the acidic solution of step (c).

Providing a solution of lifitegrast in step (a) of the process includes:
i) direct use of reaction mixture containing lifitegrast; or
ii) dissolving lifitegrast in a solvent.

Any physical form of lifitegrast may be utilized for providing the solution of lifitegrast in step (a).

Suitable solvents which can be used for dissolving lifitegrast include but are not limited to water, methanol, ethanol, isopropyl alcohol, acetone, ethyl acetate, acetonitrile, or any mixtures thereof.

The obtained solution may optionally be treated with an aqueous solution of a suitable base such as sodium hydroxide, lithium hydroxide, sodium bicarbonate ammonium hydroxide and the like.

In step (c) of the process the lifitegrast solution is treated with an aqueous solution of a suitable acid such as hydrochloric acid, sulphuric acid, orthophosphoric acid and the like.

In step (d) of the process, amorphous lifitegrast is isolated from the acidic solution of lifitegrast. The isolation can be carried out by the method known in the art such as filtration.

In another aspect, the present application provides Lifitegrast having particle sizes less than about 300 μm, or less than about 100 μm, or less than about 50 μm, or less than about 20 μm, or less than about 10 μm.

The present application provides Lifitegrast having a particle size distribution wherein the 10^{th} volume percentile particle size (D₁₀) is less than about 15 μm, the 50^{th} volume percentile particle size (D₅₀) is less than about 35 μm, and/or the 90^{th} volume percentile particle size (D₉₀) is less than about 60 μm.

For example, the present application provides Lifitegrast having a particle size distribution wherein the 10^{th} volume percentile particle size (D₁₀) is less than about 5 μm, the 50^{th} volume percentile particle size (D₅₀) is less than about 10 μm, and/or the 90^{th} volume percentile particle size (D₉₀) is less than about 20 μm. The “10^{th} volume percentile” as used herein, unless otherwise defined refers to the size of particles, below which 10% of the measured particle volume lies; “50^{th} volume percentile” as used herein, unless otherwise defined refers to the size of particles, below which 50% of the measured particle volume lies, and “90^{th} volume percentile” as used herein, unless otherwise defined refers to the size of particles, below which 90% of the measured particle volume lies.

Particle size distributions of Lifitegrast particles may be measured by any technique known in the art. For example, particle size distributions of Lifitegrast particles may be measured using light scattering equipment, such as, for example, a Malvern Master Sizer 2000 from Malvern Instruments Limited, Malvern, Worcestershire, United Kingdom (helium neon laser source, Lifitegrast suspended in light liquid paraffin, size range: 0.01 μm to 3000 μm).

In another aspect, the present application provides a pharmaceutical composition comprising Lifitegrast prepared by the processes of the invention and a pharmaceutically acceptable carrier.

In another aspect, the present application provides a pharmaceutical composition comprising Lifitegrast containing less than 0.5% w/w of its (R)-isomer and a pharmaceutically acceptable carrier.

Lifitegrast and its impurities can be analyzed using HPLC, such as with a liquid chromatography equipped with a UV detector and the parameters described below:

| Column | YMC PACK ODS AQ, 250 mm × 4.6 mm, 3.0 μm | | | | | | | | | | |
|---|---|---|---|---|---|---|---|---|---|---|---|
| Flow | 0.8 mL/min | | | | | | | | | | |
| Load | 10.0 μL | | | | | | | | | | |
| Column oven | 45 ± 2° C. | | | | | | | | | | |
| temp | | | | | | | | | | | |
| Detection | 220 nm | | | | | | | | | | |
| Diluent | Water:Acetonitrile in the ratio of 500:500 v/v | | | | | | | | | | |
| Preparation of | 1.0 mL of orthophosphoric acid in 1000 mL of water | | | | | | | | | | |
| Mobile phase-A | | | | | | | | | | | |
| Preparation of | Mix separately Acetonitrile:Water in the ratio of 900:100 v/v. | | | | | | | | | | |
| Mobile phase-B | | | | | | | | | | | |
| Gradient | | | | | | | | | | | |
| programme | Time (min) | 0 | 8 | 15 | 20 | 35 | 40 | 55 | 65 | 65.1 | 75 |
| | % M.P-A | 92 | 92 | 70 | 55 | 55 | 50 | 25 | 25 | 92 | 92 |
| | % M.P-B | 8 | 8 | 30 | 45 | 45 | 50 | 75 | 75 | 8 | 8 |

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

Definitions

The following definitions are used in connection with the present application unless the context indicates otherwise.

Amorphous form of Lifitegrast and of the present application are characterized by its PXRD pattern. All PXRD data reported herein were obtained using Cu Kα radiation, having the wavelength 1.541 A°, and were obtained using a PANalytical, Powder X-ray Diffractometer.

The term “about” when used in the present application preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11 , preferably within the range of 9.5 to 10.5, more preferably within the range of 9.8 to 10.2, and still more preferably within the range of 9.9 to 10.1.

“Amorphous form” as used herein refers to a solid state wherein the amorphous content with in the said solid state is at least about 35% or at least about 40% or at least about 45% or at least about 50% or at least about 55% or at least about 60% or at least about 65% or at least about 70% or at least about 75% or at least about 80% or at least about 85% or at least about 90% or at least about 95% or at least about 96% or at least about 97% or at least about 98% or at least about 99% or about 100%.

All percentages and ratios used herein are by weight of the total composition and all measurements made are at about 25° C. and about atmospheric pressure, unless otherwise designated. All temperatures are in degrees Celsius unless specified otherwise. As used herein, “comprising” means the elements recited, or their equivalents in structure or function, plus any other element or elements which are not recited. The terms “having” and “including” are also to be construed as open ended. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example—1

### Preparation of compound of formula IV

5,7-dichloro-2-trityl-1,2,3,4-tetrahydroisoquinoline-6-carboxylic acid (compound of formula V, 30 gm) and dimethylformamide (150 mL) were charged into a 1000 mL round bottom flask. (1[Bis(dimethylamino)methylene]-1H-1,2,3-triazolo[4,5-b] pyridinium oxide hexafluorophosphate (HATU, 29.2 gm), triethylamine (31.1 gm) and (S)-benzyl 2-amino-3-(3-(methylsulfonyl)phenyl)propanoate (compound of formula VII, 22.53 gm) were added at 28° C. and the resulted mixture was stirred for 20 hours. Water (150 mL) was added slowly into the reaction mass and stirred for 30 minutes. The precipitation was filtered and the wet solid was washed with water (60 mL). The solid was dried under vacuum at 50° C. for 20 hours. Dry weight: 46 gm.

### Example—2

### Preparation of Lifitegrast

Compound of formula IV (30 gm), 1,4-Dioxane (150 mL) were charged into a 1000 mL round bottom flask. Aqueous sodium hydroxide (1.64 gm of NaOH in 30 mL of water) was added at 28° C. and the resulted mixture was stirred for 2 hours. Concentrated hydrochloric acid (5.4 gm) was added to the reaction mixture and stirred for 2 hours. The reaction mass was concentrated completely under vacuum. Isopropyl alcohol (150 mL) was charged into the crude and the mixture was stirred for 30 minutes. The resulted mixture was concentrated under vacuum and to the residue water (90 mL) was added and stirred for 10 minutes. A mixture of ethylacetate and toluene (72 mL of ethylacetate and 108 mL of Toluene) was added to the aqueous mass and stirred for 30 minutes. Layers separated and the organic layer was extracted with water (30 mL). the aqueous layers were combined and washed with a mixture of ethylacetate and toluene (36 mL of ethylacetate and 54 mL of Toluene). The aqueous layer containing the compound of formula II (140 mL) was used in the next step.

Benzofuran-6-carboxylic acid (compound of formula VI, 2.0 gm), N,N-Dimethylformamide (20 mL) and Triethylamine (3.74 gm) were charged into a 2000 mL RBF and the mixture was cooled to 10° C. HATU (5.63 gm) was charged into the mass and stirred for 30 minutes. Added above aqueous layer containing compound of formula II at 20° C. and stirred for 1 hour. 1N aqueous hydrochloric acid (100 mL) was added and stirred for 10 minutes. Gummy solid was formed. The aqueous layer was decanted. Sodium carbonate solution (100 mL) and ethylacetate (60 mL) were added to the gummy mass and stirred for 30 minutes. Layers separated and the aqueous layer was washed with ethylacetate (60 mL). The aqueous layer was acidified with 1N aqueous hydrochloric acid (100 mL) and stirred for 30 minutes. The precipitation was faltered and the wet solid was washed with water (20 mL). The solid was dried under vacuum at 50° C. for 3 hours to yield 1.5 gm of Lifitegrast. Purity: 94.85% by HPLC.

### Example—3

### Preparation of Compound of Formula V-A

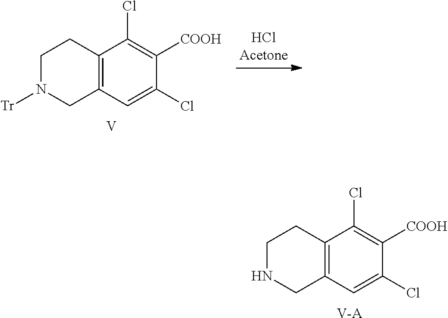

5,7-dichloro-2-trityl-1,2,3,4-tetrahydroisoquinoline-6-carboxylic acid (compound of formula V, 50 gm) and Acetone (300 mL) were charged into a 1000 mL RBF and the clear solution was stirred for 10 minutes. Concentrated hydrochloric acid (11.2 gm) was added slowly over a period of 10 minutes. The resulted mixture was stirred for 3 hours at 30° C. The precipitation was filtered and the solid was washed with acetone (250 mL). The wet compound was dried under vacuum at 55° c for 1 hour to yield 26 gm of compound of formula V-A. Purity: 95.035%.

### Example—4

### Preparation of Compound of Formula VIII

Benzofuran-6-carboxylic acid (compound of formula VII, 10 gm), acetonitrile (240 mL) and N,N-Dimethylformamide (1 mL) were charged into a 1000 mL RBF and the mixture was cooled to 0° C. Thionyl chloride (17.61 gm) was added slowly over a period of 30 minutes. The resulted mixture was stirred for 3 hours at 30° C. The reaction mixture was concentrated completely under vacuum and the resulted crude was dissolved in acetonitrile (15 mL). Compound of formula V-A (HCl salt, 17.43 gm) and N,N-Dimethylacetamide (50 mL) were charged into another RBF and stirred for 10 minutes. To the clear solution Hunig's base (DIPEA, 31.9 gm) was added. The reaction mixture was cooled to 0° C. and above acid chloride solution was added slowly over a period of 30 minutes and the reaction mixture was stirred for 30 minutes at 15° C. Water (100 mL) and aqueous hydrochloric acid were added and stirred for 10 minutes. Isopropyl alcohol (25 mL) was added and stirred for 2 hours. The precipitation was filtered and the solid was washed with water (50 mL). The wet solid and MTBE (80 mL) were charged into 500 mL RBF and stirred for 30 minutes. The precipitation was filtered and the solid was washed with MTBE (50 mL). The wet solid and isopropyl alcohol (75 mL) were charged into 500 mL RBF and stirred for 30 minutes. The precipitation was filtered and the solid was washed with isopropyl alcohol (30 mL). The wet compound was dried under vacuum at 50° C. for 3 hours to yield 19 gm of compound of formula VIII. Purity: 93.3 by HPLC.

### Example—5

### Purification of Compound of Formula VIII

Ethylacetate (80 mL) and compound of formula VIII (18 gm) were charged into a 500 mL RBF and stirred for 10 minutes. 5% Sodium bicarbonate solution was charged into the mass and stirred for 10 minutes at 28° C. Layers were separated and the aqueous layer was washed with ethylacetate. The aqueous layer was treated with charcoal and filtered through a hyflow bed. The aqueous layer was charged into another 1000 mL RBF and aqueous hydrochloric acid was added slowly by stirring the aqueous layer. The resulted mixture was stirred for 1 hour at 28° C. The precipitation was filtered and washed with water. The wet compound was slurried in isopropyl alcohol and the suspension was filtered and washed with isopropyl alcohol. The wet compound was dried under vacuum at 60° C. Dry weight: 17 g. Purity: 98.015% by HPLC.

### Example—6

### Preparation of Compound of Formula VIII

5,7-dichloro-2-trityl-1,2,3,4-tetrahydroisoquinoline-6-carboxylic acid (compound of formula V, 1.0 kg) and DMF (5 I) were charged into a 10 I round bottom flask and stirred for 15 minutes. Hydroxybenzotriazole hydrate (HOBt, 0.992 kg) was added and the mixture was stirred for 30 minutes. The reaction mixture was cooled to 15° C. and N-(3-dimethylaminopropyl)-N′-ethylcarbodiimide hydrochloride (EDC.HCl, 1.655 kg) was added lot-wise at 15° C. The reaction mass was stirred for 4 hours. Water (6 I) was added to the mass at 20° C. and stirred for 1 hour. The precipitation was filtered and the wet cake was washed with water (2 I). The wet cake and DMF (13 L) were charged into another reactor and stirred for 15 minutes. 5,7-dichloro-1,2,3,4-tetrahydroisoquinoline-6-carboxylic acid hydrochloride (compound of formula V-A, 1.743 kg) and Hunig's base (DIPEA, 31.9 gm) were added at 25° C. and the resulted mixture was stirred for 3 hours. The reaction mass was filtered through hyflosupercel bed and the bed was washed with DMF (1 I). Organic layers were combined and cooled to 15° C. Aqueous HCl (35%, 1.87 I) was added slowly to the mass and stirred for 30 minutes. Water (12 I) was added slowly into the mass and stirred for 1 hour. The precipitation was filtered and the wet cake was washed with water (3 I). The solid was dried under vacuum at 55° C. for 10 hours. The solid and DMF (10 I) were charged into a round bottom flask and the mixture was stirred for 30 minutes. The precipitation was filtered and the wet cake was washed with DMF (1 I). The wet cake and water (8 I) were charged into a flask and stirred for 2 hours. The precipitation was filtered and the wet cake was washed with water (5 I). The wet cake was press dried for 2 hours. The solid was dried under vacuum at 55° C. for 5hours to yield 1.150 kg of compound of formula VIII. Purity: 99.3 by HPLC.

### Example—7

### Preparation of Compound of Formula VII-A

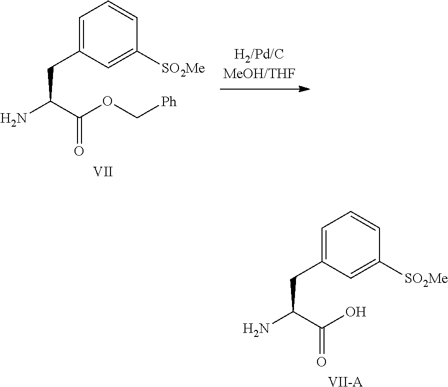

Compound of formula VII (3 gm), methanol (30 mL) and tetrahydrofuran (15 mL) were charged into a hydrogenation vessel. Palladium on carbon (0.6 gm) was added into the vessel at 28° C. and the mixture was hydrogenated using hydrogen gas (4.2 bar) and hydrogenation was continued for 2 hours at 40° C. Hydrogen pressure was released and the mass was cooled to 28° C. The mass was filtered through a hyflow bed and the bed was washed with methanol (30 mL). The solution containing the product was concentrated completely under vacuum to give 2.2 gm of the compound of formula VII-A. Purity: 97.07% by HPLC.

### Example—8

### Preparation of Lifitegrast

Compound of formula VIII (1 gm), N,N-Dimethylformamide (10 mL) and Triethylamine (1.3 gm) were charged into a 100 mL RBF. The mixture was stirred for 10 minutes at 28° C. The reaction mixture was cooled to 5° C. and 2-(3H-[1,2,3]triazolo[4,5-b]pyridin-3-yl)-1,1,3,3-tetramethylisouronium hexafluorophosphate (HATU, 1.22 gm) was charged into the mixture and the reaction mixture was stirred for 2 hours at 8° C. Compound of formula VII-A (1.29 gm) and 10 mL of DMF were added to the reaction mass at 8° C. and the mixture was stirred for 1 hour at 8° C. Pre-cooled mixture of aqueous hydrochloric acid (6 mL) and water (85 mL) were charged into the reaction mass and stirred for 30 minutes. The precipitation formed was filtered and washed with water. The wet compound and ethylacetate (20 mL) were charged into RBF and stirred for 5 minutes. Aqueous sodium bicarbonate solution (10 mL) was added to the reaction mixture and stirred for 10 minutes. Layers were separated and the aqueous layer was washed with ethylacetate (20 mL). The combined aqueous layer was acidified using dilute hydrochloric acid and the mixture was stirred for 30 minutes. The precipitation was fileted and washed with water. The wet solid was dried under vacuum at 60° C. Dry weight: 1.1 gm.

### Example—9

### Purification of Lifitegrast.

Lifitegrast (1.0 gm) and acetonitrile (10 mL) were charged into RBF. The mixture was heated to 45° C. and stirred for 10 minutes. The acetonitrile layer from the gummy mass was taken into another RBF and Dicyclohexylamine (0.31 gm) was added at 43° C. The mixture was stirred for 10 minutes and the suspension was filtered and the solid was washed with acetonitrile (5 mL). The wet solid, ethylacetate (20 mL) and aqueous sodium bicarbonate solution (30 mL) were charged into another RBF and stirring started. 50% NaOH solution (5 mL) was added and the resulted mixture was stirred for 10 minutes. Layers were separated and the aqueous layer was washed with ethylacetate (20 mL). 1N aqueous hydrochloric acid (50 mL) was charged into another RBF and above aqueous layer was added slowly to the aqueous HCl solution and stirred for 10 minutes. The precipitation was filtered and the solid was washed with water. The wet solid was dried under vacuum at 50° C. Dry weight 0.65 gm. Purity: 98.35% by HPLC. (R)-isomer content: 0.03% by HPLC.

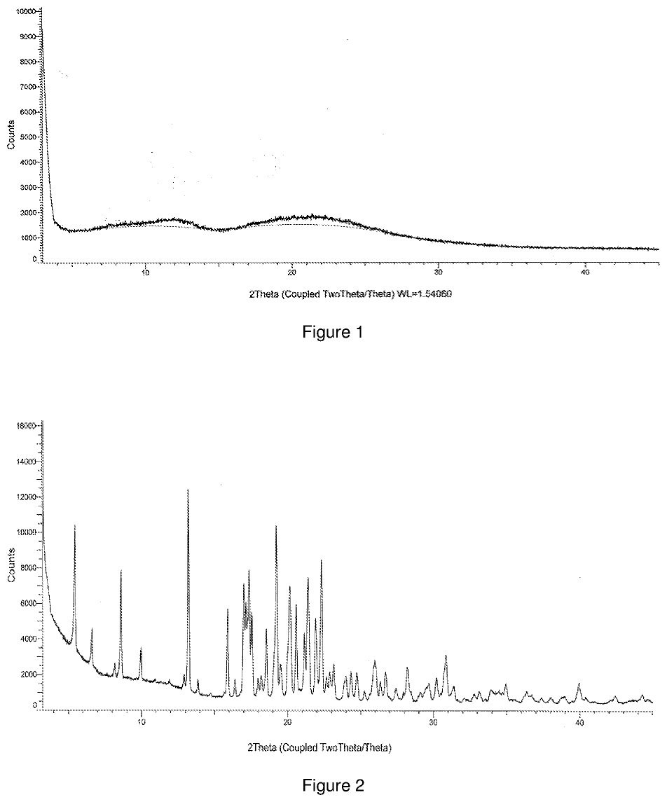

PXRD, as shown in FIG. 1, represents non-crystalline form (amorphous form).

### Example—10

### Preparation of Lifitegrast Dicyclohexylamine Salt

Compound of formula VIII (prepared in example-6, 1.0 kg) and DMF (6 I) were charged into a reactor and the mixture was stirred for 30 minutes. Hydroxybenzotriazole hydrate (HOBt, 0.412 kg) was added and the mixture was stirred for 30 minutes. The reaction mass was cooled to −5° C. and N-(3-dimethylaminopropyl)-N′-ethylcarbodiimide hydrochloride (EDC.HCl, 0.520 kg) was added lot-wise at −5° C. The reaction mass was stirred for 5 hours at 0° C. DMF (4 I), triethylamine (1.426 kg), and water (2.5 I) were added to the mass and stirred for 30 minutes. (S)-2-amino-3-(3-(methylsulfonyl)phenyl)propanoic acid hydrochloride (Compound of formula VII-A, 0.695 kg) and the reaction mixture was stirred for 8 hours at 25° C. The reaction mass was filtered and the filtrate and ethylacetate (15 I), 10% aqueous sodium chloride solution (16.5 I) were added and stirred for 30 minutes. Aqueous HCl (1.710 I) was added to the mass and stirred for 30 minutes. Organic layer was separated and the aqueous layer was extracted with ethylacetate (7 I×2). Combined the organic layers and washed with 5% aqueous sodium chloride solution (10.5 I). The organic layer and DMF (4 I) weer charged into a reactor. A solution dicyclohexylamine in ethylacetate (0.65 kg of dicyclohexylamine in 2 I ethylacetate) was added to the mass in lot-wise and the mixture was stirred for 8 hours at 25° C. The reaction mixture was filtered and the wet cake was washed with ethylacetate (6 I) and the wet cake was press dried for 3 hours. The wet solid was dried under vacuum at 55° C. Dry weight 1.55 kg

The lifitegrast dicyclohexylamine salt, ethylacetate (10 I) and DMF (4 I) were charged into a reactor and the mixture was stirred for 2 hours at 45° C. The precipitation was filtered and press dried the wet cake. The wet solid was dried under vacuum at 55° C. Dry weight 1.35 kg. Purity: 99.63% by HPLC. PXRD is shown in FIG. 2.

### Example—11

### Preparation of Lifitegrast

Lifitegrast dicyclohexylamine salt (1.0 kg), ethylacetate (16 I) and water (4 I) were charged into a reactor and stirred for 15 minutes. Orthophosphoric acid (88%, 0.314 kg) was added to the mass and stirred the mixture for 4 hours. Layers were separated. Organic layer, water (2.5 I) and Orthophosphoric acid (88%, 0.052 kg) were charged into a reactor and stirred for 1 hour. Layers separated and the organic layer was washed with water (5 I×3). Aqueous NaOH solution (0.063 kg of NaOH in 6 I of water) added to the organic layer and the mixture was stirred for 1 hour at 30° C. Layers were separated and aqueous HCl (0.523 kg of HCl in 4 I of water) was added to the organic layer and stirred for 2 hours. The precipitation was filtered and the wet solid was press dried for 2 hours. The wet solid was dried under vacuum at 55° C. Dry weight 0.605 kg. Purity: 99.71% by HPLC. PXRD is similar to FIG. 1.

### Example—12

### Preparation of Crystalline Lifitegrast

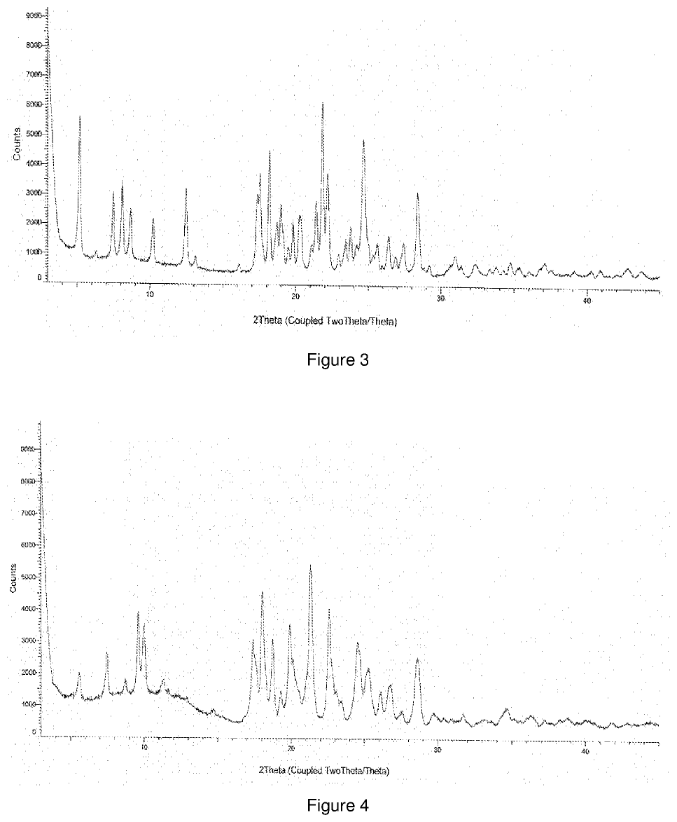

Lifitegrast dicyclohexylamine salt (10 gm), ethylacetate (150 mL) and water (50 mL) were charged into a 1000 mL round bottom flask and stirred for 10 minutes. Orthophosphoric acid (88%, 2.460 gm) was added to the mass and stirred the mixture for 30 minutes. Layers were separated. Organic layer, water (50 mL) and Orthophosphoric acid (88%, 0.307 gm) were charged into a round bottom flask and stirred for 20 minutes. Layers separated and the organic layer was washed with water (50 mL). Aqueous NaCI solution (5 mg of NaCl in 50 mL of water) added to the organic layer and the mixture was stirred for 15 minutes at 30° C. Layers were separated and basic carbon was added to the organic layer and stirred for 20 minutes. The organic layer was filtered through hyflow bed and washed with ethylacetate (20 mL). The filtered organic layer (total 160 mL) was divided into two parts (part A (80 mL) and part B (80 mL)).
Part-A: The part A ethylacetate layer was stirred for 15 minutes and 50 mg of seed crystals were added and stirred for 3 hours at 30° C. Cyclohexane (50 mL) was added and the mixture was heated to 45° C. and stirred for 3 hours. The precipitation was cooled to 28° C. and stirred for 2 hours. The precipitation was filtered and the wet solid was washed with cyclohexane (50 mL). The solid was dried under vacuum at 55° C. for 2 hours. Dry weight: 2.5 gm. PXRD is shown in FIG. 3.
Part-B: The part B ethylacetate layer was concentrated under vacuum at 50° C. Methyl ethyl ketone (15 mL) was added to the residue and distilled under vacuum at 50° C. Methyl ethyl ketone (50 mL) was added to the residue and 50 mg of seed crystals were added and stirred for 2 hours at 30° C. The precipitation was filtered and the wet solid was washed with methyl ethyl ketone (20 mL). The solid was dried under vacuum at 55° C. for 2 hours. Dry weight: 3.3 gm. PXRD is shown in FIG. 4.

1.-8. (canceled)

9. A process for preparation of Lifitegrast, comprising,
(a) reacting a compound of formula VIII with a compound of formula VII-A in presence of a base to form crude lifitegrast; 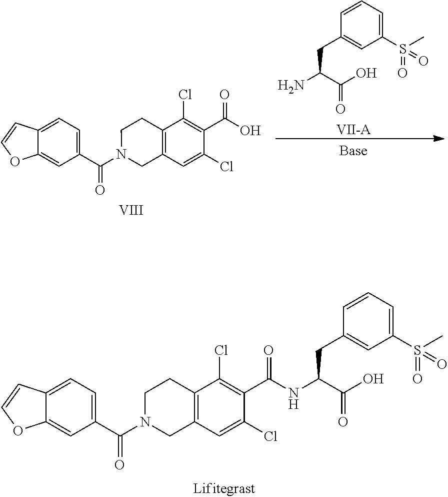
(b) treating the crude lifitegrast with a suitable amine to form an amine salt of lifitegrast; and 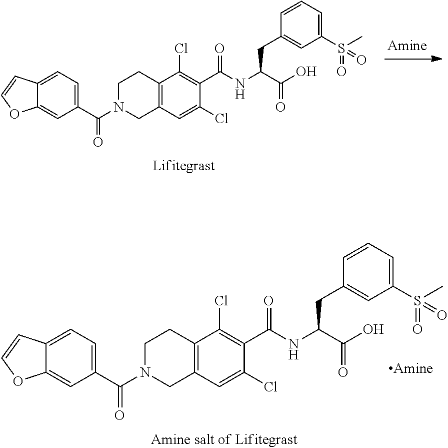 [SMILES: CS(=O)(=O)c1cccc(C[C@H](NC(=O)c2c(Cl)cc3c(c2Cl)CCN(C(=O)c2ccc4ccoc4c2)C3)C(=O)O)c1 ; CS(=O)(=O)c1cccc(C[C@H](NC(=O)c2c(Cl)cc3c(c2Cl)CCN(C(=O)c2ccc4ccoc4c2)C3)C(=O)O)c1]
(c) isolating pure lifitegrast from the amine salt of lifitegrast.

10.-12. (canceled)

13. An amine salt of lifitegrast.

14. The amine salt of lifitegrast according to claim 13, characterized in that said amine is dicyclohexyl amine.

15. The lifitegrast dicyclohexyl amine salt according to claim 14, is in crystalline form.

16. The crystalline lifitegrast dicyclohexyl amine salt according to claim 15, is characterized by a PXRD pattern of FIGS. 2.

17.-20. (canceled)

21. The process according to claim 1, wherein the lifitegrast obtained in step (c) has a purity greater than about 99.5%.

22. The process according to claim 1, wherein the lifitegrast obtained in step (c) has a purity greater than about 99.8%.

23. The process according to claim 1, wherein the lifitegrast obtained in step (c) contains less than about 0.5% of its R-isomer.

24. The process according to claim 1, wherein the lifitegrast obtained in step (c) contains less than about 0.2% of its R-isomer.

25. A process for preparation of Lifitegrast having a purity greater than about 99.5%, comprising,
(a) treating the crude lifitegrast with dicyclohexyl amine to form lifitegrast dicyclohexyl amine salt; and 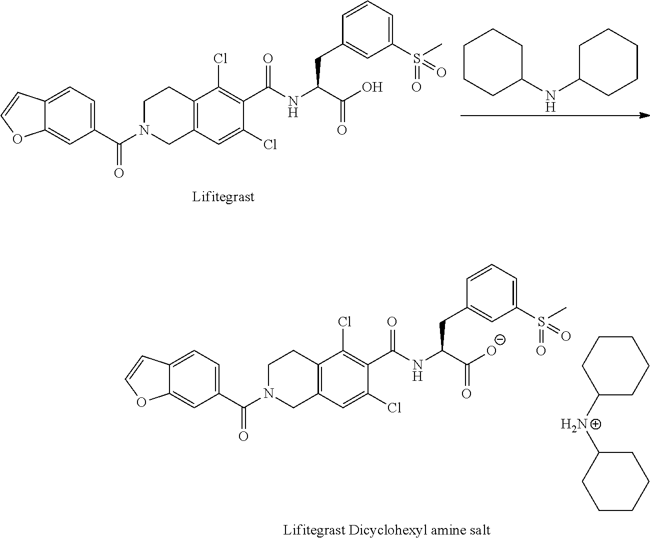
(b) isolating lifitegrast having a purity greater than about 99.5% from the lifitegrast dicyclohexyl amine salt.

26. A pharmaceutical composition comprising lifitegrast prepared by the process of claim 1, and at least one pharmaceutically acceptable carrier.
